(19) United States
(12) Patent Application Publication
(10) Pub. No.: US 2004/0243245 A1
(43) Pub. Date: Dec. 2, 2004

(54) Postero-stabilised prosthesis with non-shifting tibial stud

(76) Inventors: Sylvie Plumet, Chaumont (FR); Alexis Vouaux, Poulangy (FR)

Correspondence Address: Edward G. Greive, Renner, Kenner, Greive, Bobak, Taylor & Weber, Fourth Floor, First National Tower, Akron, OH, 44308-1456, US

(21) Appl. No.: 10/830,468

(22) Filed: Apr. 22, 2004

(30) Foreign Application Priority Data
Apr. 24, 2003 (FR) 0305060

(51) Int. Cl. A61F 2/38

(52) U.S. Cl. 623/20.27

(57) ABSTRACT

Tibial insert (1) of a so-called postero-stabilised prosthesis, having a tibial stud (2) projecting perpendicularly from the base of the insert, and having a face (6) turned towards the posterior side, characterised in that in transverse cross-section in the sagittal, or antero-posterior, plane, the posterior face (6) defines a curve, a so-called contact curve, which is, at least partially, in the from of a concave curve with its concavity turned towards the posterior side, a point (22) on the curve, in particular the summit, remote from the base, being more posterior than other points (25) on the curve, in particular most of the other points on the curve.

The present invention relates to a knee prosthesis, a so-called postero-stabilised prosthesis. The prosthesis has a femoral part intended to be fixed to the distal end of a femur, in particular by anchoring means, and a tibial part intended to be fixed to the proximal part of a tibia, in particular also by anchoring means. Between the tibial part and the femoral part an insert (also referred to as a meniscus) is provided which is usually made from a material which is softer than that of the femoral and tibial parts, such as polyethylene. In its upper part the insert has generally concave surfaces with which two condyles projecting from the femoral part come into sliding or rolling contact. A stud, a so-called tibial stud, projects in particular perpendicular to the base of the insert. Between the two condyles of the femoral part an opening, the so-called inter-condyle space, is formed, into which the tibial stud passes. In order to delimit this opening on the posterior side a so-called femoral stud is provided extending transversely to the tibial stud, from one condyle to the other.

During rotation or flexion of the knee the femoral stud comes into contact with the tibial stud, generally from a flexion angle of about 30°. In current prostheses beyond 90° of flexion the femoral part presents a high risk of rearward recoil possibly leading to considerable strain and ultimately dislocation of the prosthesis.

The present invention aims to overcome these disadvantages of the prior art by proposing a knee prosthesis, a so-called postero-stabilised prosthesis, which is safer and in particular has a reduced risk of dislocation especially for large degrees of flexion of more than 90°.

In accordance with the invention the knee prosthesis is as defined in claim 1.

By thus making provision that the point of contact descends as the flexion increases possible dislocation of the femoral part from the tibial part is counteracted, the femoral stud having a tendency to “stick” more and more to the tibial insert and thus to have less and less tendency to “unhook” itself from the top of the tibial stud.

Developments are defined in claims 2 to 9.

The present invention also relates to an insert as defined in claim 10.

In this prosthesis, the femoral stud, which is, for example, cylindro-circular in form, rolls or slides on the posterior face of the tibial stud as the knee flexes. In the prostheses of the prior art the point of contact between the femoral stud and the tibial stud tends to rise (i.e. move away from the base of the insert) as the knee flexes. By providing a more posterior summit on the contact curve possible unhooking at large flexion angles is counteracted and a safer prosthesis is thus obtained.

According to a preferred embodiment of the invention the contact curve has a straight segment, in particular in its intermediate part between the base and the summit.

According to one development of the invention the curve is, at least partially, of such a shape that from a given point to the summit a point on the curve is more posterior the closer it is to the summit.

With this type of curve, a so-called increasing posteriority curve, the point of contact between the femoral stud and the tibial stud will tend to descend as flexion progresses, thus reducing the risk of shifting.

The present invention also aims to provide a knee prosthesis having an insert in accordance with the invention.

In accordance with the invention the femoral part of a tibial prosthesis, a so-called postero-stabilised prosthesis, having two condyles between which extends a femoral stud of a cylindrical shape in order to define an opening intended to be entered by the tibial stud of the tibial insert, the tibial insert having concave upper surfaces in contact with the convex outer surfaces of the condyles, the contact being defined by a contact zone having a central point, is such that the curve delimiting the transverse cross-section of the femoral stud is defined so that the central point of the contact zone undergoes a translation in the posterior direction which decreases according to the angle of flexion until becoming zero for an angle of flexion of 80 to 90°, and remains zero to a maximum flexion of 120-130°.

By providing such kinematics for the condyle-insert point of contact, i.e. an absence of recoil from this point for flexion angles from 80-90° as far as complete flexion (120-135°) the risk of shifting of the femoral part and the wearing of the insert are greatly reduced. Thus a more long-lasting prosthesis is obtained which is safer and which more precisely imitates the kinematics of a natural knee.

According to a preferred embodiment of the invention the curve delimiting the transverse cross-section has at least two convex segments meeting at a so-called summit point, the point formed by the perpendicular projection from the summit to a straight segment connecting the two convex segments being closer to the posterior end of the straight segment than to the anterior end, in particular by a ratio of ⅓ to ⅙.

According to a preferred embodiment of the invention the convex segments of the curve delimiting the transverse cross-section of the femoral stud correspond substantially to segments of the curve defined by the transverse cross-section in the antero-posterior or sagittal plane of the outer surface of the condyles, to within a homothetic relationship.

A preferred embodiment of the invention will now be described given solely by way of example and with reference to the drawings in which:

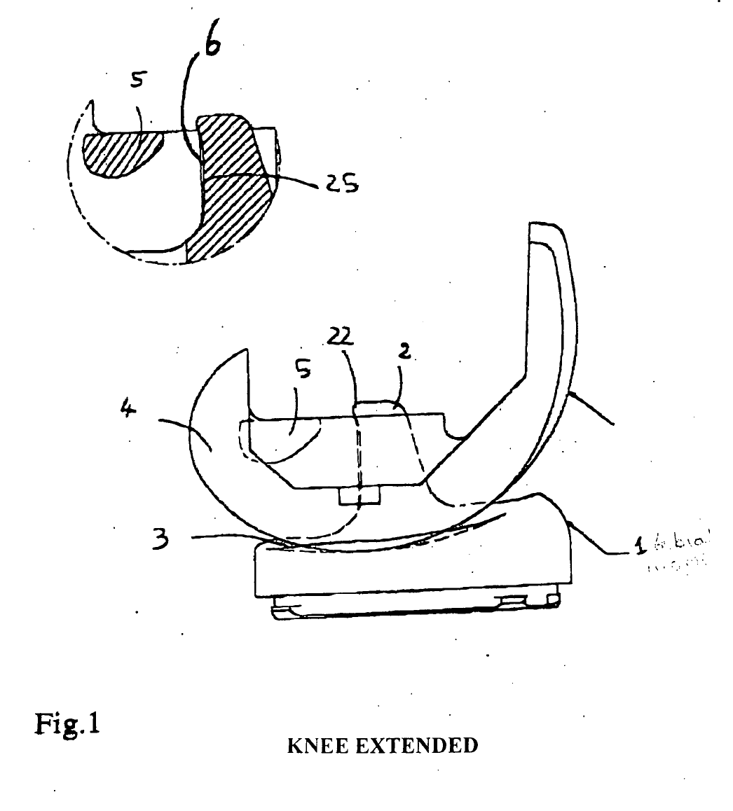

FIG. 1 illustrates the femoral part of a postero-stabilised prosthesis on a tibial insert intended to be placed on a tibial plate in the so-called antero-posterior plane when the knee is in the extended position (flexion at 0°);

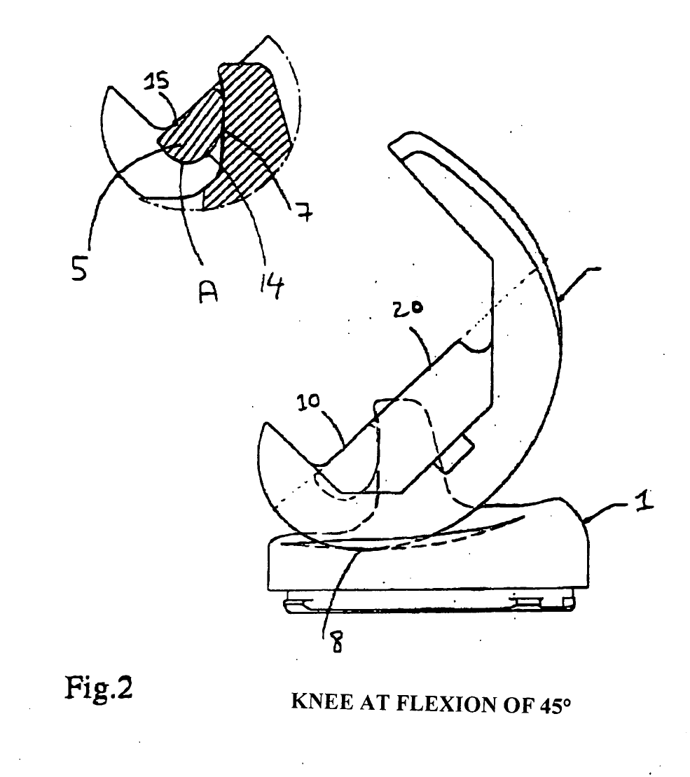

FIG. 2 illustrates the femoral part of FIG. 1 for a flexion of 45°;

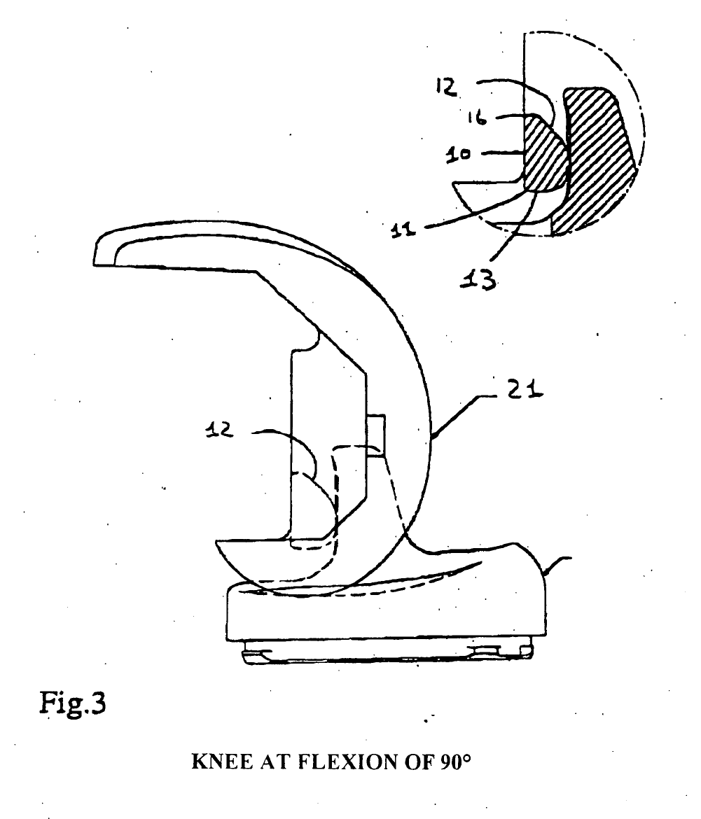

FIG. 3 illustrates the femoral part of FIG. 1 for a flexion of 90°;

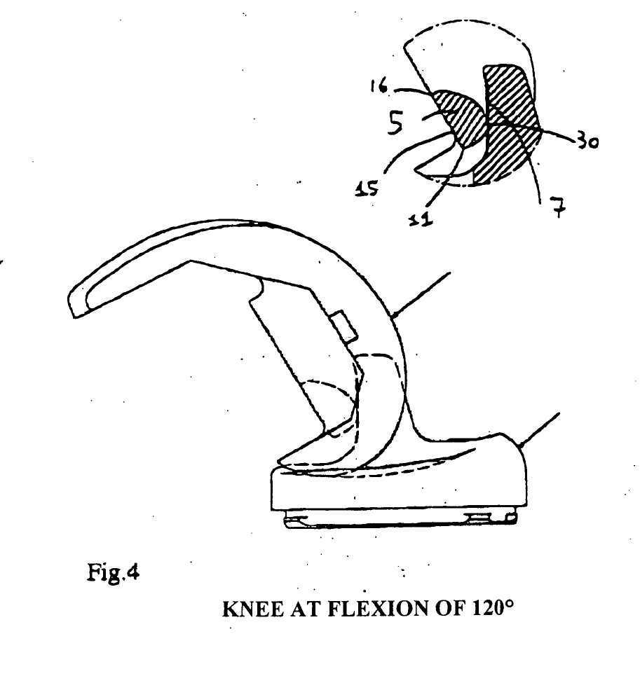

FIG. 4 illustrates the femoral part of FIG. 1 for a flexion of 120°.

FIG. 1 shows, in the antero-posterior or sagittal plane, i.e. in the plane defined by the longitudinal axes of the femur and of the tibia when the knee is flexed, a tibial insert 1 made from polyethylene, having a tibial stud 2 projecting in the vertical direction from a base of the insert, the base having two upper contact surfaces 3 with which the outer surfaces of the condyles 4 are in contact.

Between the two right and left condyles 4 (only one being shown in the figures which are cross-sectional views) an inter-condyle space is formed through which the tibial stud 2 passes. A femoral stud 5 extends from one condyle to the other in the medio-lateral direction (perpendicular to the plane of the figures). When the knee is in the extended position, the tibial stud and the femoral stud are spaced apart from each other.

From a flexion of 30° the femoral stud comes into contact with a posterior face 6 of the tibial stud. Each condyle is in contact with the insert at a contact zone, having a central point 8.

The transverse cross-section of the femoral stud is selected by taking account of the shape of the condyles, the contact surfaces of the insert and of the posterior face of the tibial stud so that the point 8 is fixed for any flexion angle between 80 to 90°0 and 130°.

In particular in accordance with a preferred embodiment the transverse cross-section of the femoral stud is formed by a straight segment 10 having a posterior end 11 and an anterior end 16, from which ends two segments of curves 12 and 13, posterior and anterior, extend. The two segments of curves are convex and in particular in this case they are arcs of a circle. They meet at a summit 14. The perpendicular projection 15 from the summit 14 to the straight section 10 is located closer to the posterior end 11 than to the anterior end 16. In particular the ratio of the distance from 15 to 11 with respect to the distance from 15 to 16 is between ⅓ and ⅙.

In particular, as shown by the figures, the shape of the closed curve 10-12-13 corresponds, to within a homothetic relationship, to the shape of the outer surface 21 of the condyles below the horizontal when the knee is in the extended position supplemented by a horizontal segment 20 (partially shown in dashed lines in the figure).

At its summit the tibial stud has a sort of tip 22 projecting in the posterior direction. The posterior face 6 of the femoral stud is dish-shaped, the bottom 25 of the dish being substantially flat and extending over substantially the whole vertical extent of the stud.

In transverse cross-section in the figure the posterior face 6 defines a contact curve.

The summit point 22 is more posterior than all the points of the tibial stud from the start of the straight segment 25 to the summit 22. Furthermore, from the point of inflexion 26 the points on the curve are more posterior the closer they are to the summit 22.

Thus the shape of the posterior surface of the tibial stud has been selected so that the contact point 30 between the femoral stud and the tibial stud descends as the angle of flexion increases, in particular in the region of large flexion angles, greater than 80-90°.

As also shown by the FIG. 4, for example, the straight segment 25 between the base and the summit 22 is perpendicular to the base of the tibial insert, in particular to the lower face of the insert which rests on the tibial plate.

1 Full knee prosthesis comprising a femoral part having a femoral stud, a tibial part and an insert, the insert being interposed between the femoral part and the tibial part and having a tibial stud projecting from a base of the insert and able to contact the femoral stud along a contact curve in the sagittal or antero-posterior plane, the shapes and arrangement of the tibial stud and of the femoral stud being such that the point of contact between the femoral stud and the tibial stud descends on the contact curve as the relative flexion of the tibial part with respect to the femoral part progresses.

2 Prosthesis as claimed in claim 1, characterised in that the tibial stud has a face (6) turned towards the posterior side, in transverse cross-section in the sagittal or antero-posterior plane, the posterior face (6) defining the contact curve which is, at least partially, in the form of a concave curve with its concavity turned towards the posterior side, a point (22) on the curve, in particular the summit, remote from the base, being more posterior than other points (25, 26) on the curve, in particular most of the other points on the curve.

3 Prosthesis as claimed in claim 1, characterised in that contact curve has a straight segment (26).

4 Prosthesis as claimed in claim 3, characterised in that the straight segment is disposed in the intermediate part between the base and the summit.

5 Prosthesis as claimed in claim 3, characterised in that the straight segment is substantially perpendicular to the base of the insert.

6 Prosthesis as claimed in claim 1, characterised in that the curve, at least in part, is of such a shape that from a given point to the summit, a point on the curve is more posterior the closer it is to the summit.

7 Full knee prosthesis as claimed in claim 1, the femoral part having two condyles between which a cylindrical femoral stud extends in order to define an opening intended to be entered by the so-called tibial stud projecting from the tibial insert, the tibial insert having concave upper surfaces in contact with the convex outer surfaces of the condyles, the contact defining a contact zone having a central point, characterised in that the curve delimiting the transverse cross-section of the femoral stud is defined so that the central point of the contact zone undergoes a rearward translation (posterior recoil) which decreases according to the angle of flexion until it becomes zero for an angle of flexion of 80 to 90°, and remains zero to a maximum flexion of 120-130°.

8 Full knee prosthesis as claimed in claim 7, characterised in that the curve delimiting the transverse cross-section has two segments which are convex, in particular arcs of a circle, meeting at a so-called summit point, the point formed by the perpendicular projection from the summit to a straight segment connecting the two segments being closer to the posterior end of the straight segment than to the anterior end, in particular by a ratio of ⅓ to ⅙.

9 Full knee prosthesis as claimed in claim 8, characterised in that the convex segments of the curve delimiting the transverse cross-section of the femoral stud correspond substantially to segments of the curve defined by the transverse cross-section in the antero-posterior plane of the outer surface of the condyles.

10 Tibial insert (1) intended to be used in a full knee prosthesis as claimed in any one of the preceding claims, the tibial insert having a tibial stud projecting from a base, the tibial stud having a face (6) turned towards the posterior side, in transverse cross-section in the sagittal or antero-posterior plane, the posterior face (6) defining the contact curve which is, at least partially, in the from of a concave curve with its concavity turned towards the posterior side, a point (22) on the curve, in particular the summit, remote from the base, being more posterior than other points (25, 26) on the curve, in particular most of the other points on the curve, characterised in that the contact curve has a straight segment (25) disposed in the intermediate part between the base and the summit, the straight segment being substantially perpendicular to the base of the insert.
